SEQUENCE LISTING (2 sequences)

SEQ ID NO: 1 - DNA, 1471 bp, Bacillus pumilus
gacgaacgctggcggcgtgcctaatacatgcaagtcgagcgaacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtg

SEQ ID NO: 2 - DNA, 3441 bp, Bacillus pumilus
atgtttcaagatatatccccaattgaggattttactggtaacctttctcttgaattcattgattacagcctaggggatcctaagtatcctgtagcagaatcaaaagaacgtgatgtaacttactctgctccactaagagtgaaagttcgtttaattaacaaagaaactggagaagtaaaagaccaagatgtgttcatgggtgatttcccaatcatgacagacacaggtacttttatcattaacggtgcggaacgtgtaatcgtttctcagttagtacgttctccaagtgtatatttcagtggtaaagtagacaaaaacggtaaaaaaggttttactgcgactgtcattccaaaccgtggcgcatggttagaatacgaaactgatgcgaaggatgtagtctatgtacgcatcgatcgcacacgtaagttgccggttacggttcttttgcgtgctctcggcttcagctctgatcaagagattcttgacctcattggcgagaatgaatacttacgcaacacgctggaaaaagacaatacagagaatgcggataaagcacttctcgaaatctacgagcgcctccgtcctggagagccgccaactgttgaaaatgcgaaaagcctgctagactctcgcttcttcgatccgaagagatatgacctagcaaatgttggacgctacaagattaataaaaagcttcatattaaaaatagactgttcaatcaaaaattggctgaaacgttagttgaccctgaaacaggtgaaattctagcagaaaaaggtcaaattttagacagaagagttcttgataaagttcttccatacttagaaaacggcatcggatttagaaagctttatccaaatggtggcgtagtagaagatgaagtagaacttcaatctattaagatttatgcaccgtctgatcaagaaggcgagcaagtgatcaacgtgatcgggaatgcatacgtagaggaagctgtgaaaaacatcacgccttctgacatcattgcatcgatcagttacttcttcaaccttcttcatggtgtaggtgacacagatgatatcgatcaccttggtaaccgtcgcctgcgttctgtaggtgagcttctgcaaaaccaattccgtattggtttaagcagaatggagcgtgttgttcgtgaaagaatgtcgatccaagacacaaacacgatcacacctcagcaactgatcaacatccgtcctgtgatcgcttctatcaaagagttctttggtagctctcagctttctcagttcatggatcaaacaaacccgcttgctgaattgacgcacaaacgtcgtctgtcagcgcttgggcctggtggtttgacacgtgagcgtgcaggaatggaagttcgtgacgttcactactctcactatggccgtatgtgtccgattgaaacaccagagggtccaaacattggtttgatcaactctctatcttcatttgcaaaagtgaatcgtttcggctttattgaaacaccttaccgccgggttgatcctgaaacaggtaaagtgacaccgagaatcgattacttaactgctgatgaagaggataactacgttgtagcacaagcgaatgctatattaggtgaagatggttcgtttatcgatgataatatcattgctcgtttcagaggggaaaacaccgttgttcctcgaaaccgtgttgactacatggacgtttcgccaaagcaggttgtttctgcagcgacagcatgtatcccattcttagaaaacgatgactctaaccgtgctctaatgggagcgaacatgcaacgtcaggctgtgcctttgatgcagccagaatcaccgattgttggtacaggtatggagtatgtatcaggtaaagactctggtgctgctgtcatctgccgttacccaggtgttgtagaacgtgttgaagcaaaaaatatttgggttcgccgctatgaagatgtcgacggacaacaagtcaaaggaaacctagacaaatacagcttgctgaaatttgtccgctctaaccaagggacttgctacaaccagcgtccaatcgtaagtgttggagatgaagtcgtaaaaggagaaatccttgcagacggtccttcaatggaaaaaggtgaattggctctaggacgtaacgttatggtcggcttcatgacttgggacggttataactacgaggatgcgatcatcatgagtgagcgccttgtaaaagatgacgtctacacatctattcatattgaagaatatgaatcagaagctcgtgatacaaagcttggaccggaagaaatcactcgtgatattccaaacgttggggaagatgctttgcgcaaccttgacgagcgtggaatcatccgtattggtgcagaagtaaaagacggagaccttcttgtagggaaagtaacgcctaaaggtgtaacagaactaacagctgaagaacgtctattacatgcaatcttcggtgaaaaggctcgtgaagtacgtgatacatctctacgtgttccacacggcggcggcggaattatccacgacgtcaaagtctttaaccgtgaagatggagatgaattacctccaggtgttaaccagttagtccgcgtatacatcgttcagaagcgtaaaatttctgaaggtgacaaaatggctggacgacatggtaacaaaggggttatctctaaaatccttccagaagaagatatgccgtatcttccagatggaacaccgattgatatcatgttaaaccctctaggggtaccatctcgtatgaacatcggtcaggtacttgagcttcatatgggtatggctgcacgttaccttggcatccacattgcgtcaccagtatttgatggtgcgcgtgaggaagatgtttgggaaacgcttgaagaagcaggtatgtcccgtgatgcgaaaacagtcctttatgacggtcgaactggtgaaccattcgataaccgtgtatcagtcggaatcatgtacatgatcaaactggctcacatggttgacgacaaacttcacgctcgttcaactggaccatactcacttgttacgcagcagccacttggcggtaaagcacagtttggtggtcagcgtttcggagagatggaagtatgggcacttgaagcttacggtgcagcatacacacttcaagagatcttaacagttaaatcggatgacgtggtgggtcgtgtgaaaacatacgaagccatcgtcaaaggggataacgtccctgaaccaggtgtccctgaatcattcaaagtgttaatcaaagaacttcaaagtttaggtatggatgtcaaaatcctatctggcgatgaagaagagatagaaatgagagatttagaagacgatgaggaaacgaagaaagcagacggattagcgttatctaatgacgaagatgctgcagacctcgcacctgtcgatcttgaacgtgacgcagtcacaaaagaatag

(19) United States
(12) Patent Application Publication
(10) Pub. No.: US 2016/0183535 A1
(43) Pub. Date: Jun. 30, 2016

(54) BACILLUS PUMILUS RTI279 COMPOSITIONS AND METHODS OF USE FOR BENEFITING PLANT GROWTH

(71) Applicant: FMC CORPORATION, Philadelphia, PA (US)

(72) Inventors: Safiyh Taghavi, Chapel Hill, NC (US); Daniel van der Lelie, Chapel Hill, NC (US); Mark R. Walmsley, Morristown, NJ (US)

(21) Appl. No.: 14/870,810

(22) Filed: Sep. 30, 2015

Related U.S. Application Data
(60) Provisional application No. 62/171,568, filed on Jun. 5, 2015.
(60) Provisional application No. 62/097,229, filed on Dec. 29, 2014.

(51) Int. Cl. A01N 63/00; A01N 43/36; C12R 1/07; A01N 43/653; C12N 1/20; A01N 37/36; A01N 47/44

(52) U.S. Cl. CPC A01N 63/00; A01N 37/36; A01N 43/36; A01N 47/44; A01N 43/653; C12N 1/20; C12R 1/07

(57) ABSTRACT

Compositions and methods include a new strain of Bacillus pumilus having plant growth promoting activity. In particular aspects, compositions containing the Bacillus pumilus strain can be applied alone or in combination with one or both of chemical agents or other microbial agents to benefit plant growth and to confer protection against and/or control plant disease.

### CROSS REFERENCE TO RELATED APPLICATIONS

This application claims the benefit of U.S. provisional application No. 62/097,229 filed Dec. 29, 2014 and U.S. provisional application No. 62/171,568 filed Jun. 5, 2015, the disclosures of which are each hereby incorporated by reference in their entireties.

### TECHNICAL FIELD

The presently disclosed subject matter relates to compositions comprising an isolated strain of Bacillus pumilus for application to plants, plant seeds, and the soil surrounding plants to benefit plant growth.

### BACKGROUND OF THE INVENTION

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microrganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) can be safer and more biodegradable than synthetic fertilizers and pesticides.

Fungal phytopathogens, including but not limited to Botrytis spp. (e.g. Botrytis cinerea), Fusarium spp. (e.g. F. oxysporum and F. graminearum), Rhizoctonia spp. (e.g. R. solani), Magnaporthe spp., Mycosphaerella spp., Puccinia spp. (e.g. P. recondita), Phytopthora spp. and Phakopsora spp. (e.g. P. pachyrhizi), are one type of plant pest that can cause servere economic losses in the agricultural and horticultural industries. Chemical agents can be used to control fungal phytopathogens, but the use of chemical agents suffers from disadvantages including high cost, lack of efficacy, emergence of resistant strains of the fungi, and undesireable environmental impacts. In addition, such chemical treatments tend to be indiscriminant and may adversely affect beneficial bacteria, fungi, and arthropods in addition to the plant pathogen at which the treatments are targeted. A second type of plant pest are bacterial pathogens, including but not limited to Erwinia spp. (such as Erwinia chrysanthemi), Pantoea spp. (such as P. citrea), Xanthomonas (e.g. Xanthomonas campestris), Pseudomonas spp. (such as P. syringae) and Ralstonia spp. (such as R. soleacearum) that cause servere economic losses in the agricultural and horticultural industries. Similar to pathogenic fungi, the use of chemical agents to treat these bacterial pathogens suffers from disadvantages. Viruses and virus-like organisms comprise a third type of plant disease-causing agent that is hard to control, but to which bacterial microorganisms can provide resistance in plants via induced systemic resistance (ISR). Thus, microorganisms that can be applied as biofertilizer and/or biopesticide to control pathogenic fungi, viruses, and bacteria are desirable and in high demand to improve agricultural sustainability. A final type of plant pathogen includes plant pathogenic nematodes and insects, which can cause severe damage and loss of plants.

Some members of the species Bacillus have been reported as biocontrol strains, and some have been applied in commercial products (Kloepper, J. W., et al., 2004, Phytopathology Vol. 94, No. 11, 1259-1266). For example, strains currently being used in commercial biocontrol products include: Bacillus pumilus strain QST2808, used as active ingredient in SONATA and BALLAD-PLUS, produced by BAYER CROP SCIENCE; Bacillus pumilus strain GB34, used as active ingredient in YIELDSHIELD, produced by BAYER CROP SCIENCE; Bacillus subtilis strain QST713, used as the active ingredient of SERENADE, produced by BAYER CROP SCIENCE; Bacillus subtilis strain GBO3, used as the active ingredient in KODIAK and SYSTEM3, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA and PONCHO-VOTIVO, produced by BAYER CROP SCIENCE. In addition, Bacillus strains currently being used in commercial biostimulant products include: Bacillus amyloliquefaciens strain FZB42 used as the active ingredient in RHIZOVITAL 42, produced by ABiTEP GmbH, as well as various other Bacillus subtilis species that are included as whole cells including their fermentation extract in biostimulant products, such as FULZYME produced by JHBiotech Inc.

The presently disclosed subject matter provides microbial compositions and methods for their use in benefiting plant growth.

### SUMMARY OF THE INVENTION

In one embodiment of the present invention, a composition for benefiting plant growth is provided including a biologically pure culture of Bacillus pumilus strain RT1279 deposited as ATCC No. PTA-121164, or a mutant thereof having all the identifying characteristics thereof, present in an amount suitable to benefit plant growth.

In one embodiment of the present invention, a coated plant seed is provided, the plant seed coated with a composition comprising spores of a biologically pure culture of Bacillus pumilus strain RT1279 deposited as ATCC No. PTA-121164, or mutants thereof having all the identifying characteristics thereof, present in an amount suitable to benefit plant growth.

In one embodiment of the present invention, a composition is provided for benefiting plant growth, the composition including a biologically pure culture of Bacillus pumilus strain RT1279 deposited as ATCC No. PTA-121164, or mutants thereof having all the identifying characteristics thereof; and an insecticide, a herbicide, a fungicide, nematicide, a bacteriocide, a plant growth regulator, a fertilizer, a microbial or a combination thereof present in an amount suitable to benefit plant growth.

In one embodiment of the present invention, a method is provided for benefiting plant growth, the method including delivering a composition including a biologically pure culture of Bacillus pumilus strain RT1279 deposited as ATCC PTA-121164, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

In one embodiment of the present invention, a method is provided for benefiting plant growth, the method including: planting a seed of the plant or regenerating vegetative/callus tissue of the plant in a suitable growth medium, wherein the seed has been coated or the vegetative/callus tissue has been inoculated with a composition comprising a biologically pure culture of a Bacillus pumilus strain RT1279 deposited as ATCC PTA-121164, or a mutant thereof having all the identifying characteristics thereof, wherein growth of the plant from the seed or the vegetative/callus tissue is benefited.

In one embodiment of the present invention, a method is provided for benefiting plant rooting, the method including: dipping a cutting of the plant in a composition and planting it in a suitable growth medium, wherein the composition comprises a biologically pure culture of a Bacillus pumilus strain RT1279 deposited as ATCC PTA-121164, or a mutant thereof having all the identifying characteristics thereof, in an amount suitable for benefiting plant rooting, wherein root formation and growth of the plant from the cutting is benefited.

In one embodiment of the present invention, a method is provided for benefiting plant growth that includes: delivering a combination of: a first composition comprising a composition comprising a biologically pure culture of a Bacillus pumilus strain RTI279 deposited as ATCC No. PTA-121164, or mutants thereof having all the identifying characteristics thereof in an amount suitable for benefiting plant growth; and a second composition comprising an insecticide, a herbicide, a fungicide, a nematicide, a bacteriocide, a plant growth regulator, a fertilizer, a microbial, or a combination thereof, in an amount suitable for benefiting plant growth, to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment of the present invention, a method is provided for benefiting plant growth that includes: delivering a composition comprising: a biologically pure culture of Bacillus pumilus strain RTI279 deposited as ATCC No. PTA-121164, or mutants thereof having all the identifying characteristics thereof, in an amount suitable for benefiting plant growth; and an insecticide, a herbicide, a fungicide, a nematicide, a bacteriocide, a plant growth regulator, a fertilizer, a microbial, or a combination thereof, in an amount suitable for benefiting plant growth to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

### BRIEF DESCRIPTION OF THE DRAWINGS

FIGS. 1A-1D show A) a schematic diagram of the genomic organization surrounding and including the osmotic stress response operon found in Bacillus pumilus strain RTI279 as compared to the corresponding regions for two Bacillus pumilus reference strains, ATCC7061 and SAFR-032 according to one or more embodiments of the present invention; B) A legend showing the gene name abbreviations; C) A legend indicating the percentage degree of amino acid identity of the proteins encoded by the genes of the RTI279 strain as compared to the two reference strains (the exact percent identity is represented numerically underneath each arrow symbol in (A)); and D) an enlarged version of the osmotic stress response operon inset from (A).

FIG. 2 shows images showing the positive effects on root hair development in soybean seedlings after inoculation of seed with Bacillus pumilus strain RT1279 at B) 1.04×10⁶ CFU/ml, C) 1.04×10⁵ CFU/ml, and D) 1.04×10⁴ CFU/ml after 7 days of growth as compared to untreated control A) according to one or more embodiments of the present invention.

FIG. 3 shows images showing the positive effects on yield in squash plants where drip irrigation was used to apply 2.5×10¹² CFU/hectare of B. pumilus RT1279 spores at the time of planting, and again 2 weeks later according to one or more embodiments of the present invention. A) Untreated control plants, and B) plants treated with RT1279 spores at 2.5×10¹² CFU/ha RT1279 by drip irrigation.

### DETAILED DESCRIPTION OF THE INVENTION

The terms “a,” “an,” and “the” refer to “one or more” when used in this application, including the claims. Thus, for example, reference to “a plant” includes a plurality of plants, unless the context clearly is to the contrary.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

In certain embodiments of the present invention, compositions and methods are provided for benefiting plant growth and conferring protection against or controlling plant pathogenic infection. A plant-associated bacterium, identified as belonging to the species Bacillus pumilus, was isolated from the rhizosphere soil of merlot vines growing at a vineyard in NY and subsequently tested for plant growth promoting properties. More specifically, the isolated bacterial strain was identified as a new strain of Bacillus pumilus through sequence analysis of highly conserved 16S rRNA and rpoB genes (see EXAMPLE 1). The 16S RNA sequence of the new bacterial isolate (designated “RTI279”) was determined to be identical to the 16S rRNA gene sequence of eight other known strains of B. pumilus. In addition, it was determined that the rpoB sequence of RTI279 has a high level of sequence similarity to the known B. pumilus SAFR-032 strain (i.e., 99% sequence identity); however, there is a 47 nucleotide difference on the DNA level, indicating that RTI279 is a new strain of B. pumilus. The strain of B. pumilus RTI279 was deposited on 17 Apr. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121164.

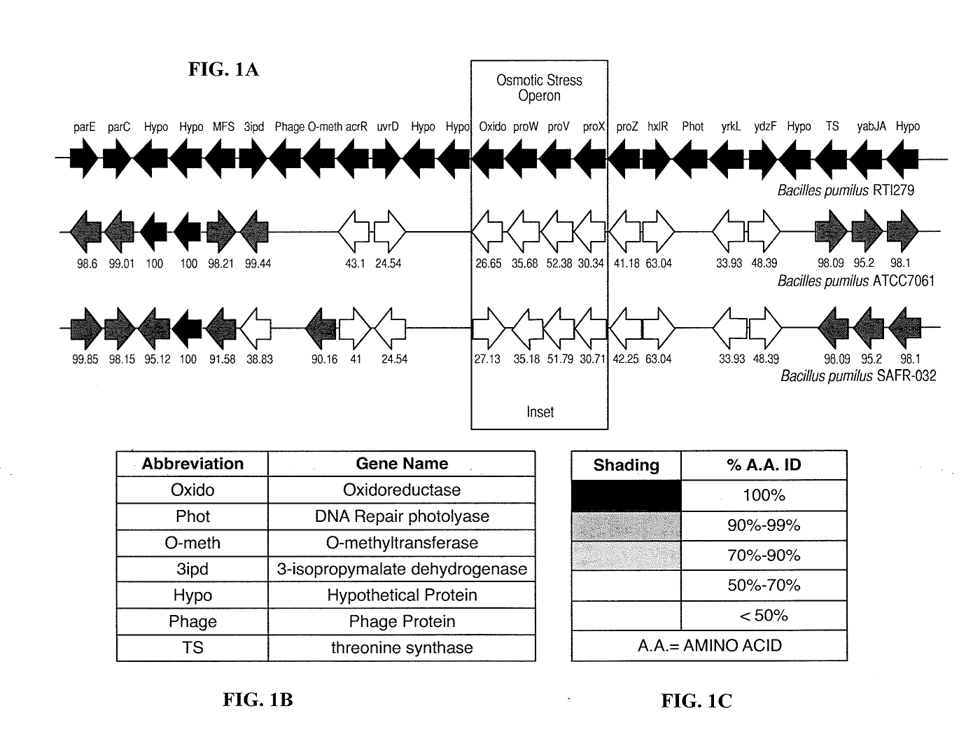

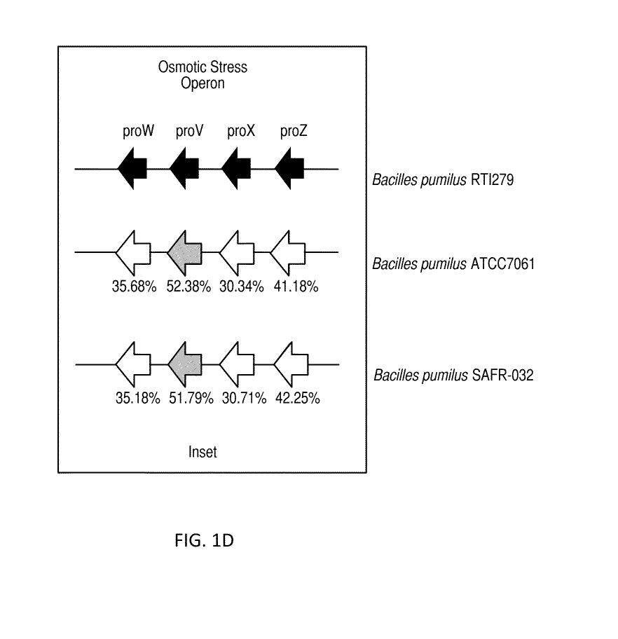

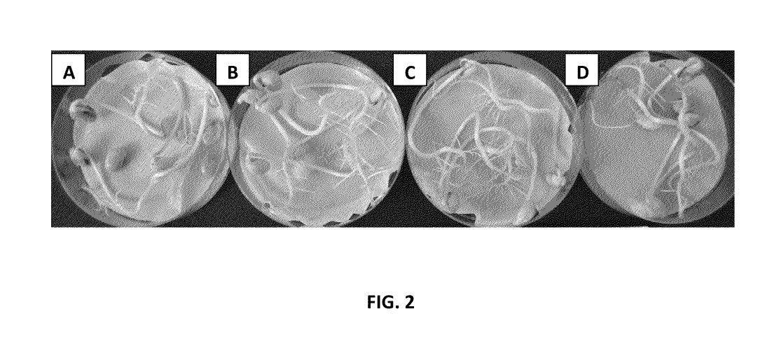

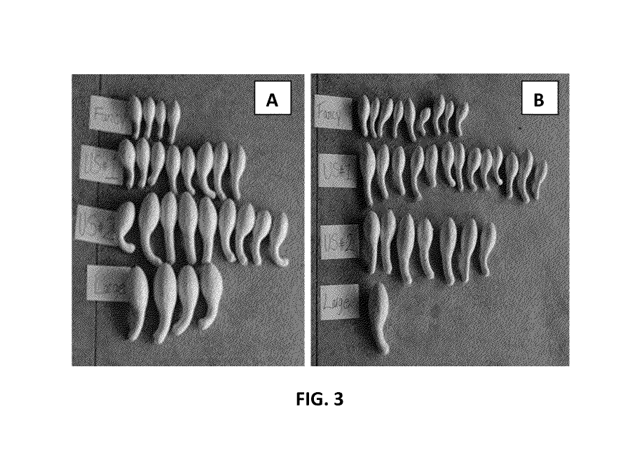

Further sequence analysis of the genome of the RTI279 Bacillus pumilus strain revealed that the strain has genes related to osmotic stress response for which homologues are lacking in other closely related B. pumilus strains (see EXAMPLE 2). This is illustrated in FIGS. 1A-1D which show a schematic diagram of the genomic organization surrounding and including the osmotic stress response operon found in Bacillus pumilus RTI279 and the corresponding region for two known Bacillus pumilus reference strains, ATCC7061 and SAFR-032. It can be observed from FIG. 1 that there is a high degree of sequence identity in the genes from the 3 different strains in the regions surrounding the osmotic stress response operon, but only a low degree of sequence identity within the operon (i.e., less than 55% within the osmotic stress response operon but greater than 90% in the surrounding regions). The low degree of sequence similarity in the region of the osmotic stress response operon between the newly identified RTI279 strain and the known Bacillus pumilus strains was unexpected and indicates that the RTI279 strain can possess properties beneficial to plant growth under conditions of osmotic stress such as drought, low moisture, and/or osmotic stress due to application of liquid fertilizer. Experiments were performed to determine the growth promoting activity of the Bacillus pumilus RTI279 strain in various plants and are provided in FIGS. 2-3 and in EXAMPLES 3-10 herein below.

For example, a positive effect of inoculation with the Bacillus pumilus RTI279 strain on early plant growth and vigor in wheat is described in EXAMPLE 3. Inoculation of wheat seeds for 2 days with Bacillus pumilus RTI279 and subsequent planting and growth in a greenhouse resulted in an 8.7% increase in dry weight of the seedlings after 13 days over non-inoculated controls. A similar result was observed for inoculation of corn seeds with Bacillus pumilus RTI279 in a greenhouse experiment (EXAMPLE 4). After 42 days, plants resulting from the RTI279 inoculated seeds showed an 8% increase in fresh shoot biomass, a 16.3% increase in dry shoot biomass, and a 6.7% increase in height as compared to non-inoculated controls.

The antagonistic properties of the Bacillus pumilus isolate RTI279 against several major plant pathogens is described in EXAMPLE 5 and phenotypic traits such as phytohormone production, acetoin and indole acetic acid (IAA), psychrophilic growth at 10° C., and nutrient cycling of the strain are described in EXAMPLE 6. The psychrophilic growth at 10° C. can be an important phenotype as it may enable the bacteria to grow and be metabolically active during the early spring when the soil temperature can often be around 10° C. such that the bacteria may benefit plant growth at lower temperatures.

EXAMPLE 7 describes positive effects of inoculation/coating of seed from a variety of plants with vegetative cells and spores of the Bacillus pumilus RTI279 strain on seed germination and root development and architecture. Positive effects were also observed on rooting of plant cutting after dipping in a composition containing RTI279. As an illustration, FIGS. 2A-2D are images of soy showing the positive effects on root hair development after inoculation by vegetative cells of RTI279 at (B) 1.04×10⁶ CFU/ml, (C) 1.04×10⁵ CFU/ml, and (D) 1.04×10⁴ CFU/ml after 7 days of growth as compared to untreated control (A). The data show that addition of the RTI279 cells stimulated formation of fine root hairs compared to non-inoculated control seeds. Fine root hairs are important in the uptake of water, nutrients and plant interaction with other microorganisms in the rhizosphere. Similar positive effects on root hair development were observed for cucumber, wheat, soy, and tomato at concentrations of RTI279 ranging from 1.04×10⁴ to 1.04×10⁹ CFU/ml (see Table IV). Positive effects on seed germination and growth after seed treatment with RTI279 spores were observed in canola, cotton, and rice (see Table V). Dipping a Hydrangea cutting into a composition containing 10⁸ CFU/g RTI279 improved the rate of rooting compared to control cuttings (see Table VI).

EXAMPLE 8 describes positive effects on growth and yield as a result of application of RTI279 spores to the soil surrounding cucumber plants in field trials. Cucumber plants, which were irrigated at the plant roots at 3 separate 1 week intervals with a solution containing fertilizer and spores of RTI279, showed the best growth and early vigor, as visualized by canopy closure, as compared to controls and each of the commercial products PHC BIOPAK and PILATUS. In addition, irrigation with the RTI279 spores had positive effects on cucumber yield (see Table VII). Specifically, RTI279 showed an increase in yield in each of three harvest periods over the untreated check and competitive standards PHC BIOPAK (LEBANON SEABOARD, CORP, Lebanon, Pa.) and PILATUS (ARYSTA LIFESCIENCE, India) (Table VII).

EXAMPLE 9 describes positive effects of coating corn seed with spores of the B. pumilus RTI279 strain in addition to a typical chemical control. In one experiment, seed treatment was performed by mixing corn seeds with a solution containing spores of B. pumilus RTI279 and chemical control MAXIM+Metalaxyl+PONCHO 250. The untreated seed and each of the treated corn seed were planted in three separate field trials in Wisconsin and analyzed by length of time to plant emergence, plant stand, plant vigor, and grain yield in bushels/acre. Inclusion of the B. pumilus RTI279 in the seed treatment as compared to the seed treated with chemical control alone did not have a statistically significant effect on time to plant emergence, plant stand, or plant vigor, but did result in an increase of 12 bushels/acre of grain (from 231 to 243 bushels/acre) representing a 5.2% increase in grain yield. A related trial was performed as described above, except that the corn plants were challenged separately with the pathogens Rhizoctonia and Fusarium graminearum. Treatment of the seed with B. pumilus RTI279 as compared to seed treated with chemical control alone resulted in a statistically significant decrease in disease severity for Fusarium graminearum. In a separate experiment, seed treatment was performed by mixing corn seeds with a solution containing spores of B. pumilus RTI279 and chemical control Ipconazole+Metalaxyl+PONCHO 500. Nineteen trials were performed with the untreated seed and each of the treated corn seeds in 11 locations across 7 states and analyzed by grain yield in bushels/acre. Inclusion of the B. pumilus RTI279 in the seed treatment as compared to the seed treated with chemical control alone resulted in an additional increase of 3 bushels/acre of grain representing a 1.5% increase in grain yield.

EXAMPLE 10 describes the positive effects on yield in squash and turnip of drip irrigation with spores of the B. pumilus RTI279 strain. A field trial was performed for squash and turnip plants where drip irrigation was used to apply 1.5×10¹¹ or 2.5×10¹² CFU/hectare of B. pumilus RTI279 spores at the time of planting, and again 2 weeks later. As compared to control squash plants in which B. pumilus RTI279 spores were not included in the irrigation, addition of the RTI279 spores resulted in an increase in yield for both total and marketable squash. Specifically, RTI279 treated plants (application rate 2.5×10¹² CFU/hectare) resulted in an average of 36 kg of total squash of which 30 kg was marketable, as compared to 22 kg of total squash of which 17 kg was marketable for the untreated control plants (see FIG. 3A (control plants) & FIG. 3B (RTI279 at application rate 2.5×10¹² CFU/hectare)). As compared to control turnip plants in which B. pumilus RTI279 spores were not included in the irrigation, addition of the RTI279 spores at both concentrations resulted in a consistent increase in turnip yield of 67% as measured in tuber weight.

In embodiments of the present invention, compositions and methods are provided that include a new strain of Bacillus pumilus having plant growth promoting activity and designated RTI279 having ATCC Accession No. PTA-121164. The compositions and methods of the presently disclosed subject matter are useful for benefiting plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium. The compositions containing the Bacillus pumilus RTI279 strain of the present invention are useful for lowering the need for nitrogen containing fertilizers and soluble minerals, increasing the availability of plant nutrients, and competing against plant pathogens, thus increasing overall plant health and decreasing the need for chemical fungicides and pesticides. The compositions containing the Bacillus pumilus RTI279 strain can be used in combination with one or more chemical agents including, for example, insecticides, herbicides, fungicides, nematicides, bacteriocides, plant growth regulators, and fertilizers.

Beneficial plant associated bacteria, both rhizospheric and endophytic, are known to provide a multitude of benefits to host plants that range from resistance to diseases and insects pests and tolerance to environmental stresses including cold, salinity and drought stress. As the plants with inoculated plant growth promoting bacteria acquire more water and nutrient from soils, e.g. due to a better developed root system, the plants grow healthier and are less susceptible to biotic and abiotic stresses. As such the microbial compositions of the present invention can be applied alone or in combination with current crop management inputs such as chemical fertilizers, herbicides, and pesticides to maximize crop productivity. Plant growth promoting effects can translate into faster growing plants and increase above ground biomass, a property that can be applied to improve early vigor. One benefit of improved early vigor is that plants are more competitive and out-compete weeds, which directly reduces the cost for weed management by minimizing labor and herbicide-application. Plant growth promoting effects can also translate into improved root development, including deeper and wider roots with more fine roots that are involved in the uptake of water and nutrients. This property allows for better use of agricultural resources, and a reduction in water used in irrigation needs and/or fertilizer application. Changes in root development and root architecture affect the interactions of the plant with other soil-borne microorganisms, including beneficial fungi and bacteria that help the plant with nutrient uptake including nitrogen fixation and phosphate solubilization. These beneficial microbes also compete against plant pathogens to increase overall plant health and decrease the need for chemical fungicides and pesticides.

In one embodiment of the present invention, a composition for benefiting plant growth is provided including a biologically pure culture of Bacillus pumilus RTI279 deposited as ATCC No. PTA-121164, or a mutant thereof having all the identifying characteristics thereof, in an amount suitable to benefit plant growth. The Bacillus pumilus RTI279 can be in the form of spores or in the form of vegetative cells. The composition benefits plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The phrase “a biologically pure culture of a bacterial strain” refers to one or a combination of: spores of the biologically pure fermentation culture of a bacterial strain, vegetative cells of the biologically pure fermentation culture of a bacterial strain, one or more products of the biologically pure fermentation culture of a bacterial strain, a culture solid of the biologically pure fermentation culture of a bacterial strain, a culture supernatant of the biologically pure fermentation culture of a bacterial strain, an extract of the biologically pure fermentation culture of the bacterial strain, and one or more metabolites of the biologically pure fermentation culture of a bacterial strain.

The growth benefit of the plant can be exhibited by one or a combination of improved seedling vigor, improved root development, improved plant growth, improved plant health, increased yield, increased resistance to osmotic stress, or improved appearance.

The compositions and methods of the present invention are beneficial to a wide range of plants including, but not limited to, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

In one or more embodiments, the plant can include soybean, wheat, cotton, corn, tomato, squash, cucumber, grass, turf grass, ornamental plants, hydrangea, or poinsettia.

The composition can be in the form of a liquid, an oil dispersion, a dust, a dry wettable powder, a spreadable granule, or a dry wettable granule. The composition can be in the form of a liquid or an oil dispersion and the Bacillus pumilus RT1279 can be present at a concentration of from about 1.0×10⁹ CFU/ml to about 1.0×10¹² CFU/ml. The composition can be in the form of a dust, a dry wettable powder, a spreadable granule, or a dry wettable granule and the Bacillus pumilus RTI279 can present in an amount of from about 1.0×10⁹ CFU/g to about 1.0×10¹² CFU/g. The composition can be in the form of an oil dispersion and the Bacillus pumilus RTI279 can be present at a concentration of from about 1.0×10⁹ CFU/ml to about 1.0×10¹² CFU/ml. The amount of the Bacillus pumilus RTI279 suitable to benefit plant growth can range from about 1.0×10⁶ CFU/ha to about 1.0×10¹³ CFU/ha.

The composition for benefiting plant growth including a biologically pure culture of the Bacillus pumilus RTI279 can be in a form of a planting matrix. The planting matrix can be in the form of a potting soil.

The composition can further include one or a combination of a microbial or a chemical insecticide, fungicide, nematicide, bacteriocide, herbicide, plant extract, plant growth regulator, or fertilizer present in an amount suitable to benefit plant growth and/or to confer protection against a pathogenic infection in a susceptible plant. The insecticide can include bifenthrin. The nematicide can include cadusafos. The insecticide can include bifenthrin and clothianidin. The composition can be formulated as a liquid and the insecticide can include bifenthrin or zeta-cypermethrin.

In one embodiment of the present invention, a coated plant seed is provided, the plant seed coated with a composition comprising spores of the biologically pure culture of Bacillus pumilus RTI279 deposited as ATCC No. PTA-121164, or a mutant thereof having all the identifying characteristics thereof, present in an amount suitable to benefit plant growth. The coated plant seed can include an amount of Bacillus pumilus spores ranging from about 1.0×10² CFU/seed to about 1.0×10⁹ CFU/seed.

The plant seed can include, but is not limited to, seed of a the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

The coated seed can further include one or a combination of an insecticide, a fungicide, a nematicide, a bacteriocide, a plant growth regulator, or a fertilizer present in an amount suitable to benefit plant growth. The insecticide can include bifenthrin. The nematicide can include cadusafos. The insecticide can include bifenthrin and clothianidin.

In one embodiment of the present invention, a composition is provided for benefiting plant growth, the composition including the biologically pure culture of Bacillus pumilus RT1279 deposited as ATCC No. PTA-121164, or mutant thereof having all the identifying characteristics thereof; and one or more chemical active agent including an insecticide, a herbicide, a fungicide, a nematicide, a bacteriocide, a plant growth regulator, or a fertilizer.

The composition can be in the form of a liquid, an oil dispersion, a dry wettable powder, a spreadable granule, or a dry wettable granule. The Bacillus pumilus RT1279 can be in the form of spores or in the form of vegetative cells. The composition can be in the form of a liquid or an oil dispersion and the Bacillus pumilus RT1279 can be present at a concentration of from about 1.0×10⁹ CFU/ml to about 1.0×10¹² CFU/ml. The composition can be in the form of a dust, a dry wettable powder, a spreadable granule, or a dry wettable granule and the Bacillus pumilus RT1279 can be present in an amount of from about 1.0×10⁹ CFU/g to about 1.0×10¹² CFU/g.

The insecticide can include bifenthrin. The nematicide can include cadusafos. The insecticide can include bifenthrin and clothianidin. The composition can be formulated as a liquid and the insecticide can include bifenthrin or zeta-cypermethrin.

The insecticide can be bifenthrin and the composition formulation can further comprise a hydrated aluminum-magnesium silicate, and at least one dispersant selected from the group consisting of a sucrose ester, a lignosulfonate, an alkylpolyglycoside, a naphthalenesulfonic acid formaldehyde condensate and a phosphate ester. The bifenthrin insecticide can be present at a concentration ranging from 0.1 g/ml to 0.2 g/ml. The bifenthrin insecticide can be present at a concentration of about 0.1715 g/ml. The rate of application of the bifenthrin insecticide can be in the range of from about 0.1 gram of bifenthrin per hectare (g ai/ha) to about 1000 g ai/ha, more preferably in a range of from about 1 g ai/ha to about 100 g ai/ha.

In addition, in one or more embodiments, suitable insecticides, herbicides, fungicides, and nematicides of the compositions and methods of the present invention can include the following:

Insecticides: A0) various insecticides, including agrigata, al-phosphide, amblyseius, aphelinus, aphidius, aphidoletes, artimisinin, autographa californica NPV, azocyclotin, Bacillus subtilis, Bacillus thuringiensis-spp. aizawai, Bacillus thuringiensis spp. kurstaki, Bacillus thuringiensis, Beauveria, Beauveria bassiana, betacyfluthrin, biologicals, bisultap, brofluthrinate, bromophos-e, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifos-e, cnidiadin, cryolite, cyanophos, cyantraniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric acid, iodomethane, iron, isocarbofos, isofenphos, isofenphos-m, isoprocarb, isothioate, kaolin, lindane, liuyangmycin, matrine, mephosfolan, metaldehyde, metarhizium-anisopliae, methamidophos, metolcarb (MTMC), mineral-oil, mirex, m-isothiocyanate, monosultap, myrothecium verrucaria, naled, neochrysocharis formosa, nicotine, nicotinoids, oil, oleic-acid, omethoate, orius, oxymatrine, paecilomyces, paraffin-oil, parathion-e, pasteuria, petroleum-oil, pheromones, phosphorus-acid, photorhabdus, phoxim, phytoseiulus, pirimiphos-e, plant-oil, plutella xylostella GV, polyhedrosis-virus, polyphenol-extracts, potassium-oleate, profenofos, prosuler, prothiofos, pyraclofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinernema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichoromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles, including ethiprole, fipronil, pyrafluprole and pyriprole; A5) the class of neonicotinoids, including acetamiprid, clothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chloranthraniliprole and cy-anthraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cyper-methrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, anitiperonosporic, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens (e.g., strains QST 713, FZB24, MBI600, D747), extract from Melaleuca alternifolia, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-IH-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-I H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-I H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fl uoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C16) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides:_{—} Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene (telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfual (aldehyde).

Suitable plant growth regulators of the present invention include the following: Plant Growth Regulators: D1) Antiauxins, such as clofibric acid, 2,3,5-tri-iodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations of the present invention can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In one embodiment of the present invention, a method is provided for benefiting plant growth, the method including delivering a composition including the biologically pure culture of the Bacillus pumilus RT1279 deposited as ATCC PTA-121164, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

The growth benefit of the plant can be exhibited by one or a combination of improved seedling vigor, improved root development, improved plant growth, improved plant health, increased yield, improved resistance to osmotic stress, or improved appearance.

The composition can be in the form of a liquid, an oil dispersion, a dust, a dry wettable powder, a spreadable granule, or a dry wettable granule. The Bacillus pumilus RT1279 can be in the form of spores or in the form of vegetative cells. The Bacillus pumilus RT1279 can be delivered at a rate of about 1.0×10⁶ CFU/ha to about 1.0×10¹³ CFU/ha to benefit the plant growth.

In the method, the composition can further include one or a combination of a microbial or a chemical insecticide, fungicide, nematicide, bacteriocide, herbicide, plant extract, or plant growth regulator, present in an amount suitable to benefit plant growth and/or to confer protection against pathogenic infection in the susceptible plant.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium. Delivery of the compositions of the present invention containing Bacillus pumilus RTI279 can ameliorate growth inhibitory effects the fertilizer may have on the plant.

In one embodiment of the present invention, a method is provided for benefiting plant growth, the method including: planting a seed of the plant or regenerating vegetative/callus tissue of the plant in a suitable growth medium, wherein the seed has been coated or the vegetative/callus tissue has been inoculated with a composition comprising a biologically pure culture of the Bacillus pumilus RTI279 deposited as ATCC PTA-121164, or a mutant thereof having all the identifying characteristics thereof, wherein growth of the plant from the seed or the vegetative/callus tissue is benefited.

The growth benefit of the plant can be exhibited by one or a combination of improved seedling vigor, improved root development, improved plant growth, improved plant health, increased yield, improved resistance to osmotic stress, or improved appearance.

The Bacillus pumilus RTI279 can be in the form of spores. The Bacillus pumilus RTI279 can be present in the form of spores at an amount ranging from about 1.0×10² CFU/seed to about 1.0×10⁹ CFU/seed. The composition coated on the seed can further comprise one or more of an insecticide, a fungicide, a nematicide, a bacteriocide, a plant growth regulator or a fertilizer present in an amount suitable to benefit plant growth.

The growth benefit of the plant can be exhibited by one or a combination of improved seedling vigor, improved root development, improved plant growth, improved plant health, increased yield, improved appearance, improved resistance to osmotic stress, or improved resistance to plant pathogens, or a combination thereof.

In one embodiment of the present invention, a method is provided for benefiting plant growth that includes delivering a combination of: a first composition comprising the biologically pure culture of the Bacillus pumilus RTI279 deposited as ATCC No. PTA-121164, or mutants thereof having all the identifying characteristics thereof; and a second composition including a one or a combination of a microbial or a chemical insecticide, fungicide, nematicide, bacteriocide, herbicide, plant extract, plant growth regulator, or fertilizer to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, wherein each of the first and second compositions are delivered in an amount suitable for benefiting plant growth.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The Bacillus pumilus RTI279 can be in the form of spores or in the form of vegetative cells. The amount of Bacillus pumilus RTI279 suitable for benefiting plant growth can range from about 1.0×10⁶ CFU/ha to about 1.0×10¹³ CFU/ha.

In one embodiment of the present invention, a method is provided for benefiting plant growth that includes: delivering a composition comprising: a biologically pure culture of the Bacillus pumilus RTI279 deposited as ATCC No. PTA-121164, or a mutant thereof having all the identifying characteristics thereof; and one or a combination of a microbial or a chemical insecticide, fungicide, nematicide, bacteriocide, plant growth regulator, or fertilizer to: a seed of the plant, roots of the plant, a cutting of the plant, a graft of the plant, callus tissue of the plant; soil or growth medium surrounding the plant; soil or growth medium before sowing the seed of the plant in the soil or growth medium; or soil or growth medium before planting the plant, the plant cutting, the plant graft, or the plant callus tissue in the soil or growth medium, wherein each of the Bacillus pumilus RTI279 and the one or a combination of a microbial or a chemical insecticide, fungicide, nematicide, bacteriocide, plant growth regulator, or fertilizer is present in an amount suitable for benefiting plant growth.

The plant growth benefit can be exhibited by improved seedling vigor, improved root development, improved plant health, increased plant mass, increased yield, improved appearance, improved resistance to osmotic stress, improved resistance to plant pathogens, or a combination thereof.

The method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The Bacillus pumilus RTI279 can be in the form of spores or in the form of vegetative cells. The amount of Bacillus pumilus RTI279 suitable for benefiting plant growth can range from about 1.0×10⁶ CFU/ha to about 1.0×10¹³ CFU/ha.

In one embodiment of the present invention, a method is provided for benefiting plant rooting, the method including dipping a cutting of a plant in a composition and planting it in a suitable growth medium, wherein the composition comprises a biologically pure culture of a Bacillus pumilus strain RTI279 deposited as ATCC PTA-121164, or a mutant thereof having all the identifying characteristics thereof, in an amount suitable to benefit plant rooting, wherein root formation and growth of the plant from the cutting is benefited.

The composition can be in the form of a liquid or a dry wettable powder. The Bacillus pumilus RTI279 can be in the form of spores or vegetative cells. The composition can be in the form of a dry wettable powder and the Bacillus pumilus RTI279 can be present in an amount of from about 1.0×10⁷ CFU/g to about 1.0×10⁹ CFU/g. The plant can be an ornamental plant. The plant can be a hydrangea.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Identification of a Bacterial Isolate as a Bacillus Pumilus Through Sequence Analysis

A plant associated bacterial strain, designated herein as RTI279, was isolated from the rhizosphere soil of merlot vines growing at a vineyard in Long Island, N.Y. The16S rRNA and the rpoB genes of the RTI279 strain were sequenced and subsequently compared to other known bacterial strains in the NCBI and RDP databases using BLAST. It was determined that the 16S RNA sequence of RTI279 (SEQ ID NO: 1) is identical to the 16S rRNA gene sequence of eight other strains of B. pumilus, including B. pumilus SAFR-032. This confirms that RTI279 is a B. pumilus. It was determined that the rpoB gene sequence of RTI279 (SEQ ID NO: 2) has a high level of sequence similarity to the gene in the B. pumilus SAFR-032 strain (99% sequence identity); however, there is a 47 nucleotide difference on the DNA level, indicating that RTI279 is a new strain of B. pumilus.

### Example 2

### Genes Related to Osmotic Stress Response in RTI279 Bacillus Pumilus

Further sequence analysis of the genome of the RTI279 Bacillus pumilus strain revealed that this strain has genes related to osmotic stress response, for which there are no homologues in the other closely related B. pumilus strains. This is illustrated in FIGS. 1A-1D, which illustrate a schematic diagram of the genomic organization surrounding and including the osmotic stress response operon found in Bacillus pumilus RTI279. In FIG. 1A, the top set of arrows represents protein coding regions for the RTI279 strain with relative direction of transcription indicated. For comparison, the corresponding regions for two Bacillus pumilus reference strains, ATCC7061 and SAFR-032, are shown below the RTI279 strain. Genes are identified by their 4 letter designation unless no designation could be found. If no designation could be found, the gene abbreviations are indicated in the legend shown in FIG. 1B. The degree of amino acid identity of the proteins encoded by the genes of RTI279 as compared to the two reference strains is indicated both by the degree of shading of the representative arrows (see FIG. 1C for the legend) as well as a numerical percentage identity indicated below the arrow. The inset shows the osmotic stress response operon identified in RTI279 and the percent amino acid identity to the corresponding encoded regions from the two reference strains. It can be observed from FIGS. 1A-1D that there is a high degree of sequence identity in the genes from the 3 different strains in the regions surrounding the osmotic stress operon, but only a low degree of sequence identity within the osmotic stress response operon (i.e., less than 55% within the osmotic stress operon but greater than 90% in the surrounding regions).

FIG. 1D shows an enlarged version of the osmotic stress operon inset from FIG. 1A. The 4 genes in the osmotic stress operon in the B. pumilus RTI279 strain were initially identified using RAST and their identities then refined using BLASTp as: proline/glycine betaine ABC transport permease (proW in FIG. 1D) based on 97% amino acid identity to Paenibacillus sp. FSL R5-192; proline/glycine betaine ATPase (proV in FIG. 1D) based on 97% amino acid identity to Paenibacillus sp. FSL R7-277, proline/glycine betaine ABC transport periplasmic component (proX in FIG. 1D) based on 97% amino acid identity to Paenibacillus sp. FSL R7-277; and proline/glycine betaine ABC permease (proZ in FIG. 1D) based on 93% amino acid identity to Paenibacillus sp. FSL R5-192. The organizational structure of the osmotic stress operon in RTI279 differs from the canonical operon organization, however all the genes required are present in the operon of RTI279. While the protein product of each of the 4 pro genes identified in the RTI279 strain has over 90% sequence identity with corresponding sequences in the genome of Paenibacillus strains deposited in the NCBI sequence database, there is only 30-52% sequence identity between these sequences and the corresponding regions in the B. pumilus strains most similar to the RTI279 strain. Thus, this osmotic stress operon is a novel feature for a B. pumilus strain and indicates the ability of this strain to benefit plant growth under conditions of drought, low moisture, and/or osmotic stress e.g. resulting from the application of liquid fertilizer.

### Example 3

### Growth Effects of Bacillus Pumilus Isolate RTI279 on Wheat

The effect of application of the bacterial isolate on early plant growth and vigor for wheat was determined. The experiment was performed by inoculating surface sterilized germinated wheat seeds for 2 days in a suspension of 10⁷ bacterial CFU/ml at room temperature under shaking (a control was performed without bacterial cells). Subsequently, the control and inoculated seeds were planted in 4″ pots in duplicate in sand mixture. Each pot was seeded with five seeds of wheat variety HARD RED at 1-1.5 cm depth. Pots were incubated in growth chamber at 24° C./18° C. with light and dark cycle of 14/10 hrs and watered as needed for 13 days. Dry weight was determined as a total weight per 10 seeds resulting in a total weight equal to 363 mg for the plants inoculated with the RTI279 strain versus a total weight equal to 333.8 mg for the non-inoculated control which is an 8.7% increase in dry weight over the non-inoculated control.

### Example 4

### Growth Effects of Bacillus Pumilus Isolate RTI279 in Corn

The effect of application of the bacterial isolate RTI279 on growth and vigor for corn was determined and the data are shown in Table I below. The experiment was performed by inoculating surface sterilized germinated corn seeds for 2 days in a suspension of 10⁸ CFU/ml of the bacterium at room temperature under shaking. Subsequently, the inoculated seeds were planted in 1 gallon pots filled with PROMIX BX which was limed to a pH of 6.5. For each treatment 9 pots were seeded with a single corn seed planted at 5 cm depth. Pots were incubated in the greenhouse at 22° C. with light and dark cycle of 14/10 hrs and watered twice a week as needed. After 42 days, plants were harvested and their height, fresh, and dry weight were measured and compared to data obtained for non-inoculated control plants. The results are shown below in Table I.

TABLE I
Growth promoting properties of Bacillus pumilus isolate
RTI279 on corn
Length of experiment 7 weeks
Location Greenhouse

| | Normalized | Normalized | |
|---|---|---|---|
| | Fresh Shoot | Dry Shoot | Height at |
| Treatment | Biomass | Biomass | 42 days |
| Control | 212.3 g | 16.99 g | 164.94 cm |
| 279 | 229.3 g | 19.77 g | 175.97 cm |
| % Increase | 8% | 16.3% | 6.7% |
| over control | | | |

### Example 5

### Anti-Microbial Properties of Bacillus Pumilus Isolate RTI279

The antagonistic ability of the isolate against major plant pathogens was measured in plate assays. A plate assay for evaluation of antagonism against plant fungal pathogens was performed by growing the bacterial isolate and pathogenic fungi side by side on 869 agar plates at a distance of 4 cm. Plates were incubated at room temperature and checked regularly for up to two weeks for growth behaviors such as growth inhibition, niche occupation, or no effect. The data for the antagonism activity is shown in Table II below.

TABLE II
Antagonistic properties of Bacillus pumilus isolate
RTI279 against major plant pathogens

| Anti-Microbial Assays | RTI279 |
|---|---|
| Aspergillus flavus | + |
| Botrytis cinerea | ++ |
| Erwinia carotovora | + |
| Fusarium graminearum | + |
| Fusarium oxysporum | +− |
| Fusarium virguliforme | − |
| Magnaporthe grisea | + |
| Phytophthora capsici | ++ |
| Pythium sylvatium | ++ |
| Rhizoctonia solani | ++ |
| Xanthomonas axonopodis | − |

+++ very strong activity,
++ strong activity,
+ activity,
+− weak activity,
− no activity observed

### Example 6

### Phenotypic Traits of Bacillus Pumilus RTI279

In addition to the positive effects on plant growth and antagonistic properties, various phenotypic traits were also measured for the RTI279 strain and the data are shown below in Table III. The assays were performed according to the procedures described in the text below Table III.

TABLE III
Phenotypic Assays: phytohormone production, acetoin and
indole acetic acid (IAA), and nutrient Cycling of Bacillus
pumilus isolate RTI279.

| Characteristic Assays | RTI279 |
|---|---|
| Acid Production (Methyl Red) | ++ |
| Acetoin Production (MR-VP) | +++ |
| Chitinase activity | − |
| Indole-3-Acetic Acid production | − |
| Protease activity | +++ |
| Phosphate Solubilization | + |
| Lowest growth temperature | 10° C. |
| Phenotype | Cream |

+++ very strong,
++ strong,
+ some,
+− weak,
− none observed

Acid and Acetoin Test.

20 μl of a starter culture in rich 869 media was transferred to 1 ml Methy Red-Voges Proskauer media (Sigma Aldrich 39484). Cultures were incubated for 2 days at 30 C 200 rpm. 0.5 ml culture was transferred and 50 μl 0.2 g/l methyl red was added. Red color indicated acid production. The remaining 0.5 ml culture was mixed with 0.3 ml 5% alpha-napthol (Sigma Aldrich N1000) followed by 0.1 ml 40% KOH. Samples were interpreted after 30 minutes of incubation. Development of a red color indicated acetoin production. For both acid and acetoin tests non-inoculated media was used as a negative control (Isenberg, H. D. (ed.), 2004. Clinical microbiology procedures handbook, vol. 1, 2 and 3, 2nd ed. American Society for Microbiology, Washington, D.C.).

Indole-3-Acetic Acid.

20 μl of a starter culture in rich 869 media was transferred to 1 ml 1/10 869 Media supplemented with 0.5 g/l tryptophan (Sigma Aldrich T0254). Cultures were incubated for 4-5 days in the dark at 30 C, 200 RPM. Samples were centrifuged and 0.1 ml supernatant was mixed with 0.2 ml Salkowski's Reagent (35% perchloric acid, 10 mM FeCl₃). After incubating for 30 minutes in the dark, samples resulting in pink color were recorded positive for IAA synthesis. Dilutions of IAA (Sigma Aldrich 15148) were used as a positive comparison; non inoculated media was used as negative control (Taghavi, et al., 2009, Applied and Environmental Microbiology 75: 748-757).

Phosphate Solubilizing Test.

Bacteria were plated on Pikovskaya (PVK) agar medium consisting of 10 g glucose, 5 g calcium triphosphate, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved. Zones of clearing were indicative of phosphate solubilizing bacteria (Sharma et al., 2011, Journal of Microbiology and Biotechnology Research 1: 90-95.).

Chitinase Activity.

10% wet weight colloidal chitin was added to modified PVK agar medium (10 g glucose, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved). Bacteria were plated on these chitin plates; zones of clearing indicated chitinase activity (N. K. S. Murthy & Bleakley., 2012. “Simplified Method of Preparing Colloidal Chitin Used for Screening of Chitinase Producing Microorganisms”. The Internet Journal of Microbiology. 10(2)).

Protease Activity.

Bacteria were plated on 869 agar medium supplemented with 10% milk. Clearing zones indicated the ability to break down proteins suggesting protease activity (Sokol et al., 1979, Journal of Clinical Microbiology. 9: 538-540).

Growth Profile.

An overnight culture of B. pumilus strain RT1279 was grown overnight at 30° C. A 10⁻⁶ dilution of the RT1279 culture was made, plated on 869 agar medium, and incubated at temperatures ranging from 5° C. to 37° C. Emergence and growth of individual colonies on different temperatures was monitored for 2 weeks.

### Example 7

### Effect of Bacillus Pumilus RTI279 on Seed Germination, Root Development, and Architecture

Experiments were performed to determine the effects of application of the B. pumilus RTI279 strain to seed on seed germination and root development and architecture. Experiments were performed as described below using both vegetative cells and spores of RTI279.

Vegetative Cells:

Assays with vegetative cells of RTI279 were performed using seed from corn, cotton, cucumber, soy, tomato, and wheat. RTI279 was plated onto 869 media from a frozen stock and grown overnight at 30° C. An isolated colony was taken from the plate and inoculated into a 50 mL conical tube containing 20 mL of 869 broth. The culture was incubated overnight with shaking at 30° C. and 200 RPM. The overnight culture was centrifuged at 10,000 RPM for 10 minutes. Supernatant was discarded and pellet was resuspended in MgSO₄ to wash. The mixture was centrifuged again for 10 minutes at 10,000 RPM. The supernatant was discarded and the pellet was resuspended in Modified Hoagland's solution. The mixture was then diluted to provide an initial concentration (10⁰). From this, 10⁻¹, 10⁻², 10⁻³, 10⁻⁴, and 10⁻⁵ dilutions of the RTI279 culture were made. For the experiments for each type of seed, 100 mm petri dishes were labeled with RTI279 or control, the dilution, and the date. A sterile filter paper was placed in the bottom of each dish. Five (5) to eight (8) seeds were placed in a single petri dish depending on the type of seed (e.g., larger seeds such as corn had smaller numbers of seed/plate). 5 mL of each dilution of RTI279 was added to the plates and the seeds were incubated at 21° C. Corn, cotton, cucumber, tomato, and wheat seeds were tested at the 10⁰, 10⁻¹, and 10⁻² dilutions. Soy seed was tested at the full range of dilutions. Control plates contained seeds and Modified Hoagland's solution without added bacteria. Images of the plates were taken after 4 and 7 days. Sterile DI water was added to the plates when they began to dry out. The data are shown in Table IV below. In addition, FIGS. 2A-2D are images of soy showing the positive effects on root hair development after inoculation by vegetative cells of RTI279 diluted by 10⁻³ (B), 10⁻⁴ (C), and 10⁻⁵ (D), corresponding to (B) 1.04×10⁶ CFU/ml, (C) 1.04×10⁵ CFU/ml, and (D) 1.04×10⁴ CFU/ml, respectively, after 7 days of growth as compared to untreated control (A). The data show that addition of the RTI279 cells stimulated formation of fine root hairs compared to uninoculated control seeds. Fine root hairs are important in the uptake of water, nutrients and plant interaction with other microorganisms in the rhizosphere.

TABLE IV
Seed germination assay for treatment
with vegetative cells of RTI279

| Vegetative Cells | | Dilution | | | | | |
|---|---|---|---|---|---|---|---|
| Crop | Starting CFU/ml | 10⁰ | 10⁻¹ | 10⁻² | 10⁻³ | 10⁻⁴ | 10⁻⁵ |
| Corn | 2.4 × 10⁸ | = | = | = | n.d. | n.d. | n.d. |
| Cotton | 1.04 × 10⁹ | − | − | = | n.d. | n.d. | n.d. |
| Cucumber | 1.04 × 10⁹ | + | ++ | ++ | n.d. | n.d. | n.d. |
| Soybean | 1.04 × 10⁹ | −− | −− | −− | ++ | ++ | + |
| Tomato | 1.04 × 10⁹ | + | + | + | n.d. | n.d. | n.d. |
| Wheat | 1.04 × 10⁹ | = | = | + | n.d. | n.d. | n.d. |

+++ very pronounced growth benefit,
++ strong growth benefit,
+ growth benefit,
+− weak growth benefit,
= no effect observed,
− weak inhibition,
−− strong inhibition,
n.d. not determined

Spores:

For the experiments using spores of RTI279, the strain was sporulated in 2×SG in a 14 L fermenter. Spores were collected but not washed afterwards at a concentration of 1.08×10¹⁶ CFU/mL. This was diluted down to 1.0×10⁷, 10⁶, and 10⁵ CFU/mL concentrations. A sterile filter paper was placed in the bottom of each sterile plastic growth chamber, and ten cucumber, radish and tomato seeds were placed in each container. Three mL of each dilution of RTI279 spores was added to the growth chambers, which were closed and incubated at 19° C. for 7 days, after which the seedlings were imaged. A positive effect on growth of the seedlings was confirmed by increased overall root size, number of root hairs, and shoot length of the seedlings. A positive effect of strain RTI279 was observed at the concentration of 1.08×10⁶ CFU/ml for cucumber and radish, and at the concentration of 1.0×10⁵ CFU/ml for tomato and Kentucky blue grass.

Coated Seed Treatment:

For the experiments using seed coated with a composition containing RTI279, the following was performed. Seed treatment was performed by mixing 100 seeds with 250 μl solution containing a total of 5×10⁶, 5×10⁷, or 5×10⁸ CFU of strain RTI279, resulting in an average of 5×10⁴, 5×10⁵, or 5×10⁶ cfu per seed. Seeds were also coated with the antifungal compounds Fludioxonil and Metalaxyl. For seed germination, a sterile filter paper was placed in a sterile transparent box. Approximately 6 to 10 seeds were placed on top of the filter paper using sterile forceps and evenly spaced. Subsequently, 15 milliliters of Modified Hoagland solution was added to each box. The boxes were then covered and stored in a dark place to reduce experimental variation. The crops were observed every 4 days for a total duration of 12 days for seed germination and notable differences in shoot and root growth. Modified Hoagland solution was also added periodically to ensure plant germination. The effects of the seed coating with B. pumilus RTI 279 were compared to Fludioxonil and Metalaxyl treated seeds to which no bacteria were added. The data are shown below in Table V.

TABLE V
Results of seed germination and growth after
seed treatment with RTI279.

| Seed | | | |
|---|---|---|---|
| Germination | | | |
| Assays | Concentration CFU/seed | | |
| Crop | 5 × 10⁴ | 5 × 10⁵ | 5 × 10⁶ |
| Canola | − | ++ | + |
| Corn | = | − | − |
| Cotton | − | + | − |
| Rice | ++ | ++ | = |

Effect on growth: ++ strong positive effect, + some improvement, = no effect observed, − weak inhibition

Rooting of Cuttings:

For the experiments to show improvement in the rooting of cuttings, a dipping composition containing RTI279 was used. The following experiments were performed. Hydrangea cuttings treatment was performed by giving the cuttings a fresh wound followed by dipping cuttings in talc powder containing 10⁸ CFU/g of strain RTI279. One gram of rooting solution powder was sufficient to treat 75 cuttings, resulting in an average of 1.3×10⁶ cfu per cutting. Cuttings were then stuck into peat pots filled with rooting media. This process represents the production standard of cut, dip, and stick. Planted flats were placed on a misted greenhouse bench and provided a light mist 4-6 times per hour. After five days the mist was controlled by an electronic leaf. The cuttings were observed every 5 days for a total duration of 35 days for the formation of roots and notable differences in root growth. The data are shown below in Table VI. The results show an increase in rooting of hydrangea cuttings due to the presence of RTI279 compared to the control.

TABLE VI
Results of rooting of Hydrangea cuttings
after treatment with RTI279.

| | % Rooting in function of days after treatment | | | | | |
|---|---|---|---|---|---|---|
| Hydrangea Treatment | 10 | 15 | 20 | 25 | 30 | 35 |
| untreated | 0 | 20 | 0 | 50 | 40 | 70 |
| RTI279 | 0 | 30 | 90 | 100 | 100 | 100 |

Effect on rooting in function of time is expressed as the % of 10 cuttings that show root formation.

### Example 8

### Growth Effects of Bacillus Pumilus Isolate RTI279 on Cucumber

The effect of application of the bacterial isolate RTI279 on growth and vigor in cucumber was determined in 20 day field trials. For field application per hectare, the spores are normally resuspended in 200 liter of water, which is subsequently diluted with 15,000 liter of water and evenly applied to the plants. To mimic these conditions in a smaller trial, the experiment was performed as described below.

For the first application of RTI279, which was done in drench (manual), 100 ml of formulated solution was applied around the root zone of each plant to allow for good incorporation into the soil. Specifically, the corresponding amount of spores (extrapolated from the amount per hectare: 3.7×10¹¹, 3.7×10¹⁰, and 3.7×10⁹ CFU to represent 2.5×10¹³ CFU/ha, 2.5×10¹² CFU/ha, and 1.25×10¹¹ CFU/ha, respectively) were suspended in 2.96 liters of water, to which 5.92 g yeast extract was also added. The solution was mixed well, and subsequently added to 25.8 liter of water containing liquid fertilizer, of which subsequently 100 ml was added per each plant. One and two weeks after the first application, the plants were again irrigated with a solution containing fertilizer, yeast extract, and spores of RTI279, except that the applied spore concentration was ten-times lower than used for the first application.

After 20 days, the first effects of the application of RTI279 on the early growth of cucumber were visually determined, using canopy closure as the measure. Compared to untreated plants (i.e., plants irrigated with liquid fertilizer only), no differences were observed when plants were irrigated with fertilizer and the blank formulation (only yeast extract, no spores). Improved growth and canopy closure was observed as compared to untreated- and blank-treated plants when the plants were treated with the commercial product PHC BIOPAK (LEBANON SEABOARD, CORP, Lebanon, Pa., a.i. 16.5 Million cfu/g each of Bacillus licheniformis, Bacillus megaterium, Bacillus polymyxa, Bacillus pumilus, Paenibacillus azotofixans, Bacillus subtilis) at 1.0 kg/ha, and the commercial product PILATUS (ARYSTA LIFESCIENCE, India, a.i. combination of plant extracts, fulvic acid, zinc, amino acids and inositol) at 1.0 L/ha, according to the manufacturers' instructions. However, irrigating the plants with the solution also containing spores of Bacillus pumilus RTI279 at 2.5×10¹³ CFU/ha, 2.5×10¹² CFU/ha, or 1.25×10¹¹ CFU/ha, resulted in the best growth and early vigor of cucumber, as visualized by canopy closure as compared to untreated- and blank-treated plants and each of the commercial products PHC BIOPAK and PILATUS.

In addition, the effect of application of the RTI279 strain as described above on cucumber yield was measured over three harvesting periods and the data are shown in Table VII below. The results show an increase in marketable yield across the twelve days of harvest. Data are presented as three different periods during the 12 day harvest period. Application of RTI279 showed an increase in yield in each of the three harvest periods over the untreated check (plants irrigated with fertilizer only) and competitive standards which were PHC BIOPAK and PILATUS.

TABLE VII
Results of cucumber yield after treatment of plant roots with RTI279 spores.

| | Vegetative & Fruiting | | | | Fruiting & Vegetative | | | | Fruiting & Senescence | | | |
|---|---|---|---|---|---|---|---|---|---|---|---|---|
| | Days 1-4 | | | | Days 5-8 | | | | Days 9-12 | | | |
| TREATMENTS | 1st | 2nd | 3rd | (Kg) | 1st | 2nd | 3rd | (Kg) | 1st | 2nd | 3rd | (Kg) |
| UNTREATED | 77% | 13% | 10% | (128) | 47% | 27% | 26% | (92) | 36% | 24% | 40% | (58) |
| RTI279 at | 73% | 14% | 13% | (135) | 48% | 29% | 23% | (104) | 39% | 26% | 35% | (67) |
| 2.50 × 10⁺¹² | | | | | | | | | | | | |
| cfu/ha | | | | | | | | | | | | |
| PHC BIOPAK WS | 73% | 13% | 14% | (131) | 44% | 30% | 26% | (92) | 39% | 24% | 37% | (55) |
| 1.0 Kg/HA | | | | | | | | | | | | |
| PILATUS 1.0 | 74% | 12% | 14% | (132) | 48% | 25% | 26% | (92) | 37% | 27% | 36% | (57) |
| LT/HA | | | | | | | | | | | | |

### Example 9

### Effects of Coating Corn Seed with Bacillus Pumilus Isolate RTI279

Experiments were performed to determine the effect of coating corn seed with spores of the B. pumilus RTI279 strain in addition to a typical chemical control. The effects on time to plant emergence, plant stand, plant vigor, and grain yield were measured for multiple field trials in Wisconsin. Experiments were performed as described below.

Formulations:

A B. pumilus RTI279 spore concentrate (1.0×10⁺¹⁰ cfu/ml) in water was applied at an amount of 1.0×10⁺⁵ cfu/seed.

MAXIM (SYNGENTA CROP PROTECTION, INC) was applied to seed at 0.0064 mg Al/kernel (fludioxonil).

Metalaxyl was applied to seed at 0.005 mg Al/kernel.

PONCHO 250 and PONCHO 500 (BAYER CROP SCIENCE) were applied to seed at 0.25 mg Al/kernel and 0.50 mg Al/kernel, respectively (Clothianidin).

Ipconazole was applied to seed at 0.0064 mg Al/kernel.

Treatment Application Method:

In one experiment, seed treatment was performed by mixing corn seeds with a solution containing spores of B. pumilus RTI279 and chemical control MAXIM+Metalaxyl+PONCHO 250 that resulted in an average of 1×10⁵ cfu per seed and the chemical active ingredients at the label-indicated concentrations as detailed above. The experiment was performed with untreated seed and seed treated with the chemical control alone as controls. The untreated seed and each of the treated corn seed were planted in three separate field trials in Wisconsin and analyzed by length of time to plant emergence, plant stand, plant vigor, and grain yield in bushels/acre. Using an average of the data from the three field trials, addition of the chemical control as compared to untreated seed resulted in a statistically significant increase in each of time to plant emergence, plant stand, plant vigor, and grain yield. Inclusion of the B. pumilus RTI279 in the seed treatment as compared to the seed treated with chemical control alone did not have a statistically significant effect on time to plant emergence, plant stand, or plant vigor, but did result in an increase of 12 bushels/acre of grain (from 231 to 243 bushels/acre) representing a 5.2% increase in grain yield.

A related trial was performed as described above, except that the corn plants were challenged separately with the pathogens Rhizoctonia and Fusarium graminearum. Disease severity was rated by visual inspection on a scale of 1 to 5. Treatment of the seed with B. pumilus RTI279 as compared to seed treated with chemical control alone resulted in a statistically significant decrease in disease severity for Fusarium graminearum.

In a separate experiment, seed treatment was performed by mixing corn seeds (2 different varieties were tested per trial) with a solution containing spores of B. pumilus RTI279 and chemical control Ipconazole+Metalaxyl+PONCHO 500 that resulted in an average of 1×10⁵ cfu per seed and the chemical active ingredients at the label-indicated concentrations as detailed above. Nineteen trials were performed with the untreated seed and each of the treated corn seeds in 11 locations across 7 states and analyzed by grain yield in bushels/acre. Using an average of the data from 16 of the field trials, addition of the chemical control as compared to untreated seed resulted in a statistically significant increase (9.8 bushels/acre) in grain yield. Inclusion of the B. pumilus RTI279 in the seed treatment as compared to the seed treated with chemical control alone resulted in an additional increase of 3 bushels/acre of grain representing a 1.5% increase in grain yield.

### Example 10

### Effects of Drip Irrigation with Bacillus Pumilus Isolate RTI279 on Squash and Turnip

Experiments were performed to determine the effect of drip irrigation with spores of the B. pumilus RTI279 strain on squash and turnip. The effects on plant growth and yield were determined according to the experiments described below.

A field trial was performed for squash plants where drip irrigation was used to apply 1.5×10¹¹ or 2.5×10¹² CFU/hectare of B. pumilus RTI279 spores at the time of planting, and again 2 weeks later. As compared to control plants in which B. pumilus RTI279 spores were not included in the irrigation, addition of the RTI279 spores resulted in an increase in yield for both total and marketable squash. Specifically, RTI279 treated plants (application rate 2.5×10¹² CFU/hectare) resulted in an average of 36 kg of total squash of which 30 kg was marketable, as compared to 22 kg of total squash of which 17 kg was marketable for the untreated control plants (FIG. 3A (control plants) & FIG. 3B (RTI279 at application rate 2.5×10¹² CFU/hectare)).

A similar field trial was performed in which turnip plants were drip irrigated with 2.5×10¹¹ or 2.5×10¹² CFU/hectare of B. pumilus RTI279 spores at the time of planting and again 2 weeks later. As compared to control plants in which B. pumilus RTI279 spores were not included in the irrigation, addition of the RTI279 spores at both concentrations resulted in a consistent increase in yield of 67% as measured in tuber weight.

### REFERENCES

All publications, patent applications, patents, and other references cited herein are incorporated herein by reference in their entireties.

That which is claimed:

1. A composition for benefiting plant growth, the composition comprising a biologically pure culture of Bacillus pumilus RTI279 deposited as ATCC No. PTA-121164, or a mutant thereof having all the identifying characteristics thereof, present in an amount suitable to benefit plant growth.

2. The composition of claim 1, wherein the composition is capable of benefiting plant growth when applied to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

3. The composition of claim 1, wherein the composition is in the form of a liquid, an oil dispersion, a dust, a dry wettable powder, a spreadable granule, or a dry wettable granule.

4. The composition of claim 1, wherein the composition is in the form of a liquid and the Bacillus pumilus RTI279 is present at a concentration of from about 1.0×10⁹ CFU/ml to about 1.0×10¹² CFU/ml.

5. The composition of claim 1, wherein the composition is in the form of a dust, a dry wettable powder, a spreadable granule, or a dry wettable granule and the Bacillus pumilus RTI279 is present in an amount of from about 1.0×10⁹ CFU/g to about 1.0×10¹² CFU/g.

6. The composition of claim 1, wherein the composition is in the form of an oil dispersion and the Bacillus pumilus RTI279 is present at a concentration of from about 1.0×10⁹ CFU/ml to about 1.0×10¹² CFU/ml.

7. The composition of claim 1, wherein the Bacillus pumilus RTI279 is in the form of spores.

8. The composition of claim 1, wherein the Bacillus pumilus RTI279 is in the form of vegetative cells.

9. The composition of claim 1, further comprising one or a combination of a microbial or a chemical insecticide, fungicide, nematicide, bacteriocide, herbicide, plant extract, plant growth regulator, or fertilizer present in an amount suitable to benefit plant growth and/or to confer protection against a pathogenic infection in a susceptible plant.

10. The composition of claim 9, wherein the insecticide comprises bifenthrin.

11. The composition of claim 9, wherein the nematicide comprises cadusafos.

12. The composition of claim 9, wherein the insecticide comprises bifenthrin and clothianidin.

13. The composition of claim 9, formulated as a liquid.

14. The composition of claim 13, wherein the insecticide comprises bifenthrin or zeta-cypermethrin.

15. The composition of claim 1, wherein the composition is in the form of a planting matrix.

16. The composition of claim 15, wherein the planting matrix is in the form of a potting soil.

17. A plant seed coated with a composition comprising:
spores of a biologically pure culture of Bacillus pumilus RTI279 deposited as ATCC No. PTA-121164, or a mutant thereof having all the identifying characteristics thereof, present in an amount suitable to benefit plant growth.

18. The plant seed of claim 17, wherein the composition comprises an amount of Bacillus pumilus spores from about 1.0×10² CFU/seed to about 1.0×10⁹ CFU/seed.

19. The plant seed of claim 17, wherein the seed comprises the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

20. The plant seed of claim 17, wherein the composition further comprises one or a combination of a microbial or a chemical insecticide, fungicide, nematicide, bacteriocide, herbicide, plant extract, or plant growth regulator present in an amount suitable to benefit plant growth.

21. The plant seed of claim 20, wherein the insecticide comprises bifenthrin.

22. The plant seed of claim 20, wherein the nematicide comprises cadusafos.

23. The plant seed of claim 20, wherein the insecticide comprises bifenthrin and clothianidin.

24. A composition for benefiting plant growth, the composition comprising:
a biologically pure culture of Bacillus pumilus RTI279 deposited as ATCC No. PTA-121164, or a mutant thereof having all the identifying characteristics thereof; and
one or a combination of a microbial or a chemical insecticide, fungicide, nematicide, bacteriocide, herbicide, plant extract, plant growth regulator, or fertilizer in an amount suitable to benefit plant growth.

25. The composition of claim 24, wherein the composition is in the form of a liquid or an oil dispersion and the Bacillus pumilus RTI279 is present at a concentration of from about 1.0×10⁹ CFU/ml to about 1.0×10¹² CFU/ml.

26. The composition of claim 24, wherein the composition is in the form of a dust, a dry wettable powder, a spreadable granule, or a dry wettable granule and the Bacillus pumilus RTI279 is present in an amount of from about 1.0×10⁹ CFU/g to about 1.0×10¹² CFU/g.

27. The composition of claim 24, wherein the Bacillus pumilus RTI279 is in the form of spores.

28. The composition of claim 24, wherein the Bacillus pumilus RTI279 is in the form of vegetative cells.

29. The composition of claim 24, wherein the insecticide comprises bifenthrin.

30. The composition of claim 24, wherein the nematicide comprises cadusafos.

31. The composition of claim 24, wherein the insecticide comprises bifenthrin and clothianidin.

32. The composition of claim 24, formulated as a liquid.

33. The composition of claim 32, wherein the insecticide comprises bifenthrin or zeta-cypermethrin.

34. A method for benefiting growth of a plant, the method comprising delivering a composition comprising a biologically pure culture of Bacillus pumilus RTI279 deposited as ATCC No. PTA-121164, or a mutant thereof having all the identifying characteristics thereof to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit plant growth.

35. The method of claim 34, wherein the growth benefit of the plant is exhibited by improved seedling vigor, improved root development, improved plant growth, improved plant health, increased yield, improved appearance, improved resistance to osmotic stress, or a combination thereof.

36. The method of claim 34, wherein the Bacillus pumilus RTI279 is delivered at a rate of about 1.0×10⁶ CFU/ha to about 1.0×10¹³ CFU/ha.

37. The method of claim 34, wherein the Bacillus pumilus RTI279 is in the form of spores.

38. The method of claim 34, wherein the Bacillus pumilus RTI279 is in the form of vegetative cells.

39. The method of claim 34, wherein the composition is in the form of a liquid, an oil dispersion, a dust, a dry wettable powder, a spreadable granule, or a dry wettable granule.

40. The method of claim 34, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

41. The method of claim 34, wherein the composition further comprises one or a combination of a microbial or a chemical insecticide, fungicide, nematicide, bacteriocide, herbicide, plant extract, plant growth regulator, or fertilizer present in an amount suitable to benefit plant growth and/or to confer protection against pathogenic infection in the susceptible plant.

42. The method of claim 41, wherein the insecticide comprises bifenthrin.

43. The method of claim 41, wherein the nematicide comprises cadusafos.

44. The method of claim 41, wherein the insecticide comprises bifenthrin and clothianidin.

45. The method of claim 42, wherein the composition is formulated as a liquid.

46. The method of claim 45, wherein the insecticide comprises bifenthrin or zeta-cypermethrin.

47. A method for benefiting growth of a plant, the method comprising:
planting a seed of the plant or regenerating vegetative/callus tissue of the plant in a suitable growth medium, wherein the seed has been coated or the vegetative/callus tissue has been inoculated with a composition comprising a biologically pure culture of Bacillus pumilus RTI279 deposited as ATCC PTA-121164, or a mutant thereof having all the identifying characteristics thereof, wherein growth of the plant from the seed or the vegetative/callus tissue is benefited.

48. The method of claim 47, wherein the growth benefit of the plant is exhibited by improved seedling vigor, improved root development, improved plant growth, improved plant health, increased yield, improved appearance, improved resistance to osmotic stress, or a combination thereof.

49. The method of claim 47, wherein the Bacillus pumilus RTI279 is present in the form of spores at an amount of from about 1.0×10² CFU/seed to about 1.0×10⁹ CFU/seed.

50. The method of claim 47, wherein the seed comprises the seed of monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

51. The method of claim 47, wherein the composition further comprises one or a combination of a microbial or a chemical insecticide, fungicide, nematicide, bacteriocide, herbicide, plant extract, plant growth regulator, or fertilizer present in an amount suitable to benefit plant growth.

52. The method of claim 51, wherein the insecticide comprises bifenthrin.

53. The method of claim 51, wherein the nematicide comprises cadusafos.

54. The method of claim 51, wherein the insecticide comprises bifenthrin and clothianidin.

55. The method of claim 51, wherein the composition is formulated as a liquid.

56. The method of claim 55, wherein the insecticide comprises bifenthrin or zeta-cypermethrin.

57. The method of claim 47, wherein the Bacillus pumilus RTI279 is in the form of spores.

58. The method of claim 47, wherein the Bacillus pumilus RTI279 is in the form of vegetative cells.

59. A method for benefiting plant growth, the method comprising:
delivering a combination of:
a first composition comprising a biologically pure culture of Bacillus pumilus RTI279 deposited as ATCC No. PTA-121164, or a mutant thereof having all the identifying characteristics thereof in an amount suitable for benefiting plant growth; and
a second composition comprising one or a combination of a microbial or a chemical insecticide, fungicide, nematicide, bacteriocide, herbicide, plant extract, plant growth regulator, or fertilizer in an amount suitable for benefiting plant growth,
to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

60. The method of claim 59, wherein the plant growth benefit is exhibited by improved seedling vigor, improved root development, improved plant health, increased plant mass, increased yield, improved appearance, improved resistance to osmotic stress, improved resistance to plant pathogens, or a combination thereof.

61. The method of claim 59, wherein the amount suitable for benefiting plant growth is from about 1.0×10⁶ CFU/ha to about 1.0×10¹³ CFU/ha Bacillus pumilus RTI279.

62. The method of claim 59, wherein the Bacillus pumilus RTI279 is in the form of spores.

63. The method of claim 59, wherein the Bacillus pumilus RTI279 is in the form of vegetative cells.

64. The method of claim 59, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

65. The method of claim 59, wherein the insecticide comprises bifenthrin.

66. The method of claim 59, wherein the nematicide comprises cadusafos.

67. The method of claim 59, wherein the insecticide comprises bifenthrin and clothianidin.

68. The method of claim 59, wherein the insecticide is formulated as a liquid.

69. The method of claim 68, wherein the insecticide comprises bifenthrin or zeta-cypermethrin.

70. A method for benefiting plant growth, the method comprising:
delivering a composition comprising:
a biologically pure culture of Bacillus pumilus RTI279 deposited as ATCC No. PTA-121164, or a mutant thereof having all the identifying characteristics thereof in an amount suitable for benefiting plant growth; and
one or a combination of a microbial or a chemical insecticide, fungicide, nematicide, bacteriocide, plant growth regulator, or fertilizer in an amount suitable for benefiting plant growth,
to: seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

71. The method of claim 70, wherein the plant growth benefit is exhibited by improved seedling vigor, improved root development, improved plant health, increased plant mass, increased yield, improved appearance, improved resistance to osmotic stress, improved resistance to plant pathogens, or a combination thereof.

72. The method of claim 70, wherein the Bacillus pumilus RTI279 is in the form of spores.

73. The method of claim 70, wherein the Bacillus pumilus RTI279 is in the form of vegetative cells.

74. The method of claim 70, wherein the plant comprises monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hydrangea, Hardwood cuttings, Chestnuts, Oak, Maple, sugarcane, or sugarbeet.

75. The method of claim 70, wherein the amount suitable for benefiting plant growth is from about 1.0×10⁶ CFU/ha to about 1.0×10¹³ CFU/ha Bacillus pumilus RTI279.

76. The method of claim 70, wherein the insecticide comprises bifenthrin.

77. The method of claim 70, wherein the nematicide comprises cadusafos.

78. The method of claim 70, wherein the insecticide comprises bifenthrin and clothianidin.

79. The method of claim 70, formulated as a liquid.

80. The method of claim 79, wherein the insecticide comprises bifenthrin or zeta-cypermethrin.

81. A method for benefiting plant rooting, the method comprising:
dipping a cutting of a plant in a composition and planting it in a suitable growth medium, wherein the composition comprises a biologically pure culture of a Bacillus pumilus strain RTI279 deposited as ATCC PTA-121164, or a mutant thereof having all the identifying characteristics thereof, in an amount suitable to benefit plant rooting, wherein root formation and growth of the plant from the cutting is benefited.

82. The method of claim 81, wherein the Bacillus pumilus RTI279 is in the form of spores.

83. The method of claim 81, wherein the Bacillus pumilus RTI279 is in the form of vegetative cells.

84. The method of claim 81, wherein the composition is in the form of a liquid or a dry wettable powder.

85. The composition of claim 84, wherein the composition is in the form of a dry wettable powder and the Bacillus pumilus RTI279 is present in an amount of from about 1.0×10⁷ CFU/g to about 1.0×10⁹ CFU/g.
